(19) United States
(12) Patent Application Publication
(10) Pub. No.: US 2025/0035586 A1
(43) Pub. Date: Jan. 30, 2025

(54) IMPROVEMENTS IN OR RELATING TO A METHOD OF ANALYSING A COMPONENT IN A SAMPLE

(71) Applicant: FLUIDIC ANALYTICS LIMITED, Cambridge, Cambridgeshire (GB)

(72) Inventors: Alekszej MORGUNOV, Tallinn (EE); Jan BURRI, Baar (CH); Thomas MUELLER, Baar (CH); Quentin PETER, Baar (CH); Tuomas KNOWLES, Cambridge (GB)

(73) Assignee: FLUIDIC ANALYTICS LIMITED, Cambridge, Cambridgeshire (GB)

(21) Appl. No.: 18/711,746

(22) PCT Filed: Nov. 18, 2022
(86) PCT No.: PCT/GB2022/052935

§ 371 Date: May 20, 2024

(30) Foreign Application Priority Data
Nov. 18, 2021 (GB) 2116626.9

(51) Int. Cl. G01N 27/447; G01N 33/58

(52) U.S. Cl. CPC G01N 27/44726; G01N 27/44791; G01N 33/582

(57) ABSTRACT

A method of determining the quantity of two or more species in a polydisperse sample is provided. The method comprising the steps of: performing a series of dilution steps of the polydisperse sample; adding a probe into the polydisperse sample, wherein the probe is configured to bind to two or more species to form two or more probe-target species complexes; separating two or more probe-target species complexes at each dilution step into two or more bins; determining the concentration of each separated probe-target species complex in each bin at each dilution step; and determining the quantity of each target species in the sample and/or the affinity of each target species to the probe.

The present invention relates to a method for analysing a biophysical property of a component in a sample and, in particular, a method of determining the quantity of two or more species in a polydisperse sample.

Protein-protein interactions form the basis of many biologically and physiologically relevant processes including: protein self-assembly; protein-aggregation; antibody-antigen recognition; muscle contraction and cellular communication. Nevertheless, studying protein-protein interactions, especially under physiological conditions in complex media, remains challenging. Current techniques, such as an enzyme-linked immunosorbent assay (ELISA) bead-based multiplex assay and surface plasmon resonance (SPR) spectroscopy, rely on immobilisation of one binding partner. These techniques include potential unspecific interactions with the surface, which can cause false-positive results and the Hook/Prozone effect, which causes false-negative results, thereby allowing semi-quantitative analysis only.

Microfluidic devices have been previously deployed to separate components in a fluid flow within a sample in a channel. The flow can then be split into further downstream flows where the sample is further labelled. Electrokinetic separation techniques, such as capillary zone electrophoresis (CE), capillary gel electrophoresis (CGE), capillary isoelectric focusing (CIEF), capillary isotachophoresis and micellar electrokinetic chromatography (MEKC) are powerful analytical tools commonly used to separate a plurality of components in a sample in solution. Separation of components using CE is based on the electrophoretic mobility of the component and thus CE allows for determination of purity of components in a sample.

The separated samples can then be detected optically with absorption, scattering or fluorescence measurements. Alternatively, the separated samples can be determined electrically or electrochemically as well as off chip after collection or injection into a down-stream detection module.

Working with samples containing multiple protein species can be challenging. This problem is typically solved by using an affinity probe to fish out the species of interest. In some cases, however, the affinity probe will bind to several species. For example, a blood sample might have several antibody types that binds to the same viral protein. Therefore, there is a requirement to provide a method that is capable of separating these subspecies and determine the biophysical properties of each species within the sample, including the concentration, dissociation constant, and/or size.

It is against the background that the present invention has arisen.

According to an aspect of the present invention, there is provided a method of determining the quantity of two or more species in a polydisperse sample, the method comprising the steps of: performing a series of dilution steps of the polydisperse sample; adding a probe into the polydisperse sample, wherein the probe is configured to bind to two or more species to form two or more probe-target species complexes; separating two or more probe-target species complexes at each dilution step into two or more bins; determining the concentration of each separated probe-target species complex in each bin at each dilution step; and determining the quantity of each target species in the sample and/or the affinity of each target species to the probe.

With reference to the present invention the term “quantity” is used to mean any reasonable measure of the amount of each species present. In particular, the quantity can be expressed as a concentration or as a flux or as a number of molecules.

The separation may be temporal or spatial. It may occur across a range of times such as separation times or elution times or across a physical space, such as across a channel or within a tube. In embodiments in which separation is temporal then the mechanism may be capillary electrophoresis or chromatography. In embodiments in which separation is spatial then the mechanism may be free-flow electrophoresis or centrifugation. The collection of the separated species is optional and can theoretically be done in either case.

In some embodiments, the content of each bin is one “species” which may contain one or more different type of molecule. In some embodiments, the resolution may not be sufficient to separate subtly different molecules, but can separate out major differences between molecules, which, in this context, are referred to as species.

The determination of the target species is distinct from the determination of the probe-target species complex for all circumstances other than a strong binding regimen in which all target is bound to a probe and thus detected.

Although the steps are not necessarily carried out in the order listed above, there are advantages to performing the dilution steps before adding the probe in that the same probe stock can be used and applied to each dilution. Conversely, if it is preferred it is also possible to manipulate the probe concentration so that it is neither the same in each dilution nor the same as if the probe had been added prior to the dilution series. This enables the probe concentration to be set to obtain maximum information about the species present in the polydisperse sample.

The probe must be configured to bind to two or more species in order for the method to be effective. There may be other probes present, but amongst the probes present, there must be at least one probe that binds to two or more species of interest within the polydisperse sample.

The series of dilution steps comprises a minimum of three dilutions. Preferably, one dilution is above, one is around and one is below the expected K_{D} of the species. If there is not the preferred spread of dilutions, then results will still be obtained, but the uncertainty in the values determined will be higher. Preferably, the dilutions are set out to span a factor of ×100 in target concentration. The accuracy of the results will be improved by having more than three dilutions in the series: typically 5, 6, 7, 8 or more dilutions are used to order to cover a reasonable range with reasonable accuracy.

In some embodiments, the method steps may be carried out within a diluted regimen. Within the context of the present invention the term “diluted regimen” is used to refer to any regimen that is not the strong or tight binding regimen in which the probe concentration is considerably higher than the binding affinity, K_{D} and the undiluted target species. This includes the weak binding regimen in which the probe concentration is much smaller than the binding affinity and the medium binding regimen in which the probe concentration is of the same order of magnitude as the binding affinity. In any diluted regimen, the method adds value because not all of the targets are bound. If all target is bound, then the concentration in each bin then the quantity of the target is the same as the concentration of the probe-target complex and the method is not required.

In some embodiments, the step of determining the quantity of each target species in the sample includes fitting the measured probe-target species complexes to a model including binding equilibria with one or more affinities between probes and targets, where the fitting parameters are the concentrations of each target and their affinities to the probe.

In some embodiments, the method may further comprise the step of determining the quantity of free probe present in each dilution and/or in each bin.

Within the context of the present invention, the term “free probe” is used to refer to a probe which is not bound to the target species.

In some embodiments, the same concentration of probe can be added at each dilution step.

In some embodiments, the sample containing two or more species, for example proteins or protein-protein complexes, can be incubated with an affinity probe prior to separation. The concentration of the free probe can be varied, as well as the sample dilution. A separation step can then be carried out on the sample thereafter. Using a curve fitting model, the quantitative information about the biophysical properties of the proteins and/or protein-protein interactions can be obtained, for example the concentration of the free probe and the concentration of all separated sub-species of proteins and protein complexes can be determined.

Additionally or alternatively, the concentration of the sample is known, but not the dissociation constant. In this case, several dilutions of the sample can be mixed with a known concentration of a labelled probe to produce a dilution curve. The dissociation constant can be extracted from the dilution curve.

Additionally or alternatively, both the dissociation constant and the concentration of the subspecies are unknown. This would typically require mixing of several dilutions of the sample with two or more concentration of an affinity probe. Using a curve fitting model, both the concentration of the sample and the dissociation constant can be extracted in this case.

In some embodiments, the concentration of probe added to one or more of the dilutions steps differs from the concentration of the probe added to one or more of the other dilutions steps.

In some embodiments, the method of the present invention may further comprise the step of selecting the concentration of the probe and/or selecting one or more dilutions step using a feedback mechanism based on the determined concentration of each separated probe-target species complex, the determined quantity of each target species and/or the affinity of each target species to the probe obtained in a previous experiment.

Within the context of the present invention, the choice of which sample dilution and probe concentration to use during experiments can either be considered before the experiment or updated during the experiment by using a feedback mechanism based on results from previous experiments. Choosing the most suitable dilution or probe concentration can be from a user designing the experiments, or it can be chosen automatically by using automated software after analysing the results of previous experiments. The analysis of the results may be based on Bayesian analysis.

As disclosed herein, and unless otherwise specified, the term polydisperse refers to a sample of one or more components.

In some instances, it can be particularly useful to determine the ratio between species that are bound and not bound to another molecule in order to identify the target species of interest in a polydisperse sample. For example, the polydisperse sample can be provided with an affinity probe or an immunoprobe which can have a specific affinity to a single species or multiple species of interest. The probe may be labelled. A known quantity of an affinity probe or an immunoprobe can be added to the polydisperse sample.

The method of the present invention as disclosed herein can then be used to determine the ratio between the unbound and bound affinity probe and/or the immunoprobe within the sample. This, in turn, can be used to determine the quantity and/or affinity of each of the two or more species of interest in the polydisperse sample to the affinity probe.

In some embodiments, multiple affinity probes can be used to detect and/or quantify multiple species of interest as well as determine their affinity to the multiple affinity probes.

A detector can be provided to detect the two or more species bound to a probe separated into two or more bins at each dilution step.

The detection techniques deployed may be selected, as appropriate from various different approaches to the detection of electromagnetic radiation of various types. The detection techniques may include, but are not limited to, the following;
Optical detection, across any suitable waveband, not limited to the visible, but including X-ray, UV and IR as applicable
   Fluorescence such as detecting the auto-fluorescence or intrinsic fluorescence of the species or detecting the fluorescence of a dye attached onto the species;
   Chemiluminescence;
   Absorbance;
   Scattering;
Electrical detection
   Conductivity detection;
   Charge detection;
   Electrochemical detection;
Mass measurement
Detection of radioactivity from radioactive labels

In some embodiments, the probe can be an affinity probe or an immunoprobe. Preferably, the probe is an affinity probe.

These labels can be specific to a sub-set of the species for example, labelled immuno-probes for “fishing” out targets from a complex mixture such as a blood plasma sample.

The affinity probe can be, but is not limited to, one or more of the following: an antibody, antibody fragment, a peptide, a protein scaffold, an affimer, an affibody, a single-chain antibody, a single-chain variable fragment (scvf), a small molecule, a nanobody, an aptamer or a darpin.

In some embodiments, the method of the present invention may further comprise the step of labelling the probe with a label.

Labelling the probe enables the detection of the species in each bin. Examples of labelling may include, but is not limited to, the following: Immunolabelling, such as attaching a labelled immune-probe, including antibodies, antibody fragments, single-chain antibodies, aptamers; fluorescent labels, FRET pair labelling and/or a combination thereof.

In some embodiments, the label of the probe can be a non-latent fluorescence label. Non-latent labels can be specific to a sub-set of the species for example, labelled immuno-probes for “fishing” out targets from a complex mixture such as blood plasma.

In some embodiments, the label may be at a single binding site on the probe.

In some embodiments, the label may be a fluorescent molecule.

In some embodiments, the probe may have a lower molecular weight than the target species.

In some embodiments, the separation of the two or more species can be created electrophoretically through the application of an electric field.

In some embodiments, the separation of the two or more species can be created by capillary electrophoresis.

Additionally or alternatively, the separation of two or more species can be created using, but is not limited to, one or more of the following techniques; liquid and/or gas chromatography techniques such as size-exclusion chromatography (SEC), ion-exchange chromatography (IEC), reverse-phase chromatography, hydrophobic interaction chromatography, Fast protein liquid chromatography, Mass Spec or LC-MS; asymmetric flow-field flow fractionation (AF4), hydrodynamic separation, by diffusion and/or magnetically through the application of a magnetic field.

It can be advantageous to provide two or more species in their native state because separation and analysis can be done based on the inherent biophysical properties of the species.

In some embodiments, the method may further comprise the step of identifying one or more, partially or completely, separated peaks corresponding to each species. The identification of one or more peaks corresponding to each separated species can be carried out in an electropherogram. The skilled person in the art would understand that any equivalent analysis tool or chart to the electropherogram may be used for identifying one or more separated peaks, which is associated with each of the separated species.

In some embodiments, the peaks in the electropherogram or equivalent may contain partially separated peaks corresponding to each species. In this case, the method may further comprise the step of applying a Gaussian fitting to the partially separated peaks. The Gaussian fitting of the peaks can be used for separating the species. This can enable the identification of the species where the peaks slightly overlap in the electropherogram.

In some embodiments, the species can be, but is not limited to, one or more of the following: an antibody or antibody fragment thereof, a polypeptide, a polynucleotide, a protein, a small molecule or a polysaccharide.

As disclosed in the present invention, and unless otherwise specified, the term “species” or “component” refers to a biomolecule or a biological or chemical molecule or particle. The biomolecule can be a protein, a polypeptide, a polysaccharide, a peptide, an amino acid, a small molecule, an exosome or a lipid, an antibody or an antibody fragment thereof, a nucleotide or polynucleotide such as DNA or DNA piece, RNA or mRNA, or a polysaccharide. The biomolecule may be a complex of different biomolecules.

In some embodiments, the method of the present invention may further comprise the step of removing background signal of the probe. This ensures that the background noise is reduced or eliminated in order to improve the signal resolution of the species of interest during analysis.

In some embodiments, the method of the present invention may further comprise the step of adjusting one or more parameters of the polydisperse sample to facilitate binding between the probe and the two or more species.

Each parameter as disclosed in the present invention can provide different effects to the polydisperse sample. For example, adjusting the temperature can change the thermodynamics of the species within the sample, which may affect the interactions between the probe and the species. In another example, adjusting the salt concentration may allow the study of whether the binding between the probe and the species is dominated by charged interactions.

In some embodiments, the parameters can be, but is not limited to, one or more of the following: temperature, salt concentrations, pH, and viscosity of the sample and/or surfactant concentration.

The polydisperse sample may include, but is not limited to, saliva, blood, serum, plasma, urine, cerebrospinal fluid (CSF), sperm, mucus and/or lysate samples.

The invention will now be further and more particularly described, by way of example only, and with reference to the accompanying drawings, in which:

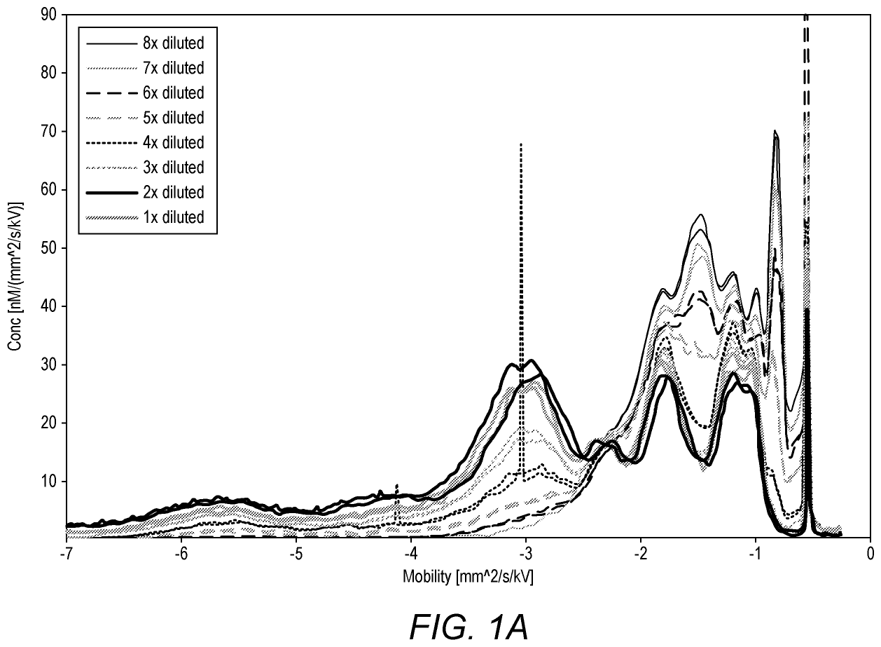

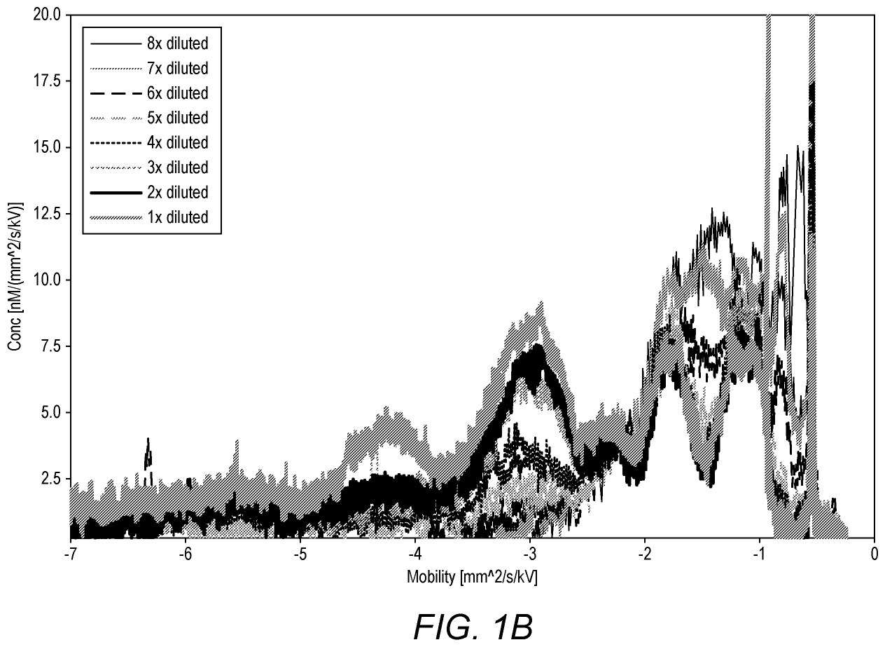

FIGS. 1A to 1B show experimentally obtained electrophoretic separations of dilution series of a polydispersed sample at different probe concentrations according to the present invention;

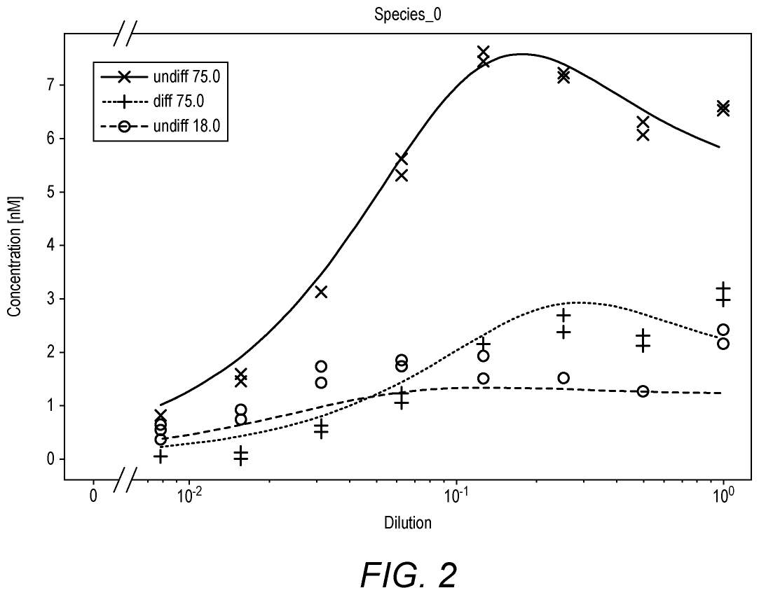

FIG. 2 shows a dilution series of one separated species in order to determine the concentration and affinity of that species to the probe;

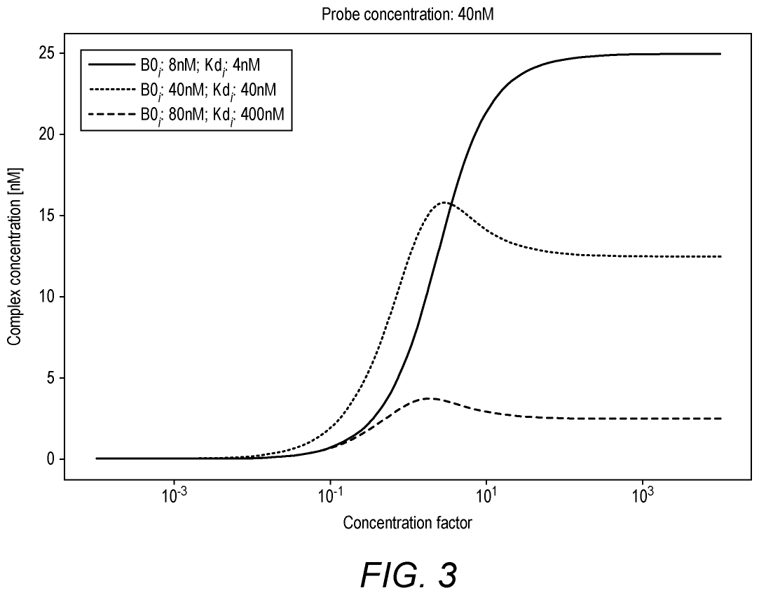

FIG. 3 provides a graph showing the concentration of complexes;

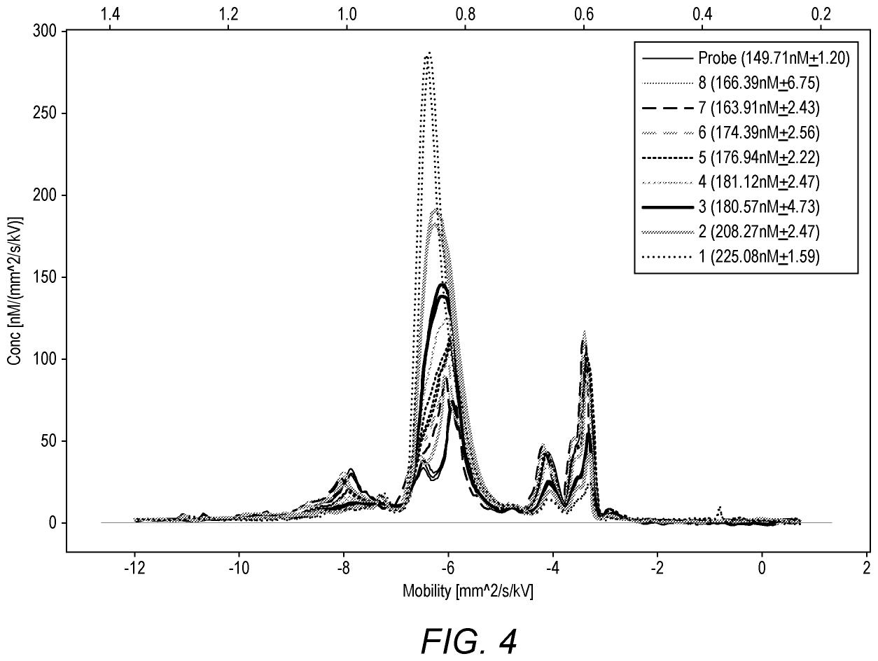

FIG. 4 provides a graph showing the separation of species at different sample concentrations and constant probe concentration; and

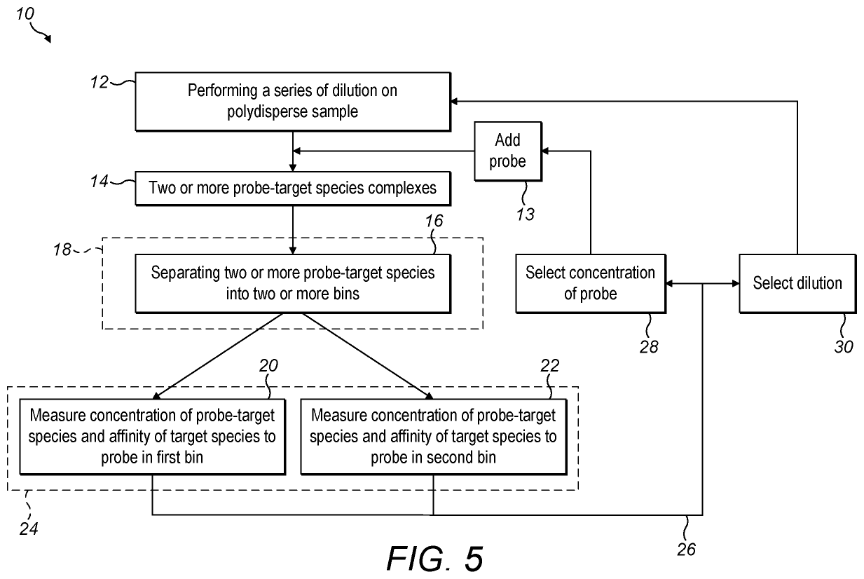

FIG. 5 shows a flowchart illustrating the workflow of the present invention.

An exemplary workflow may be provided to determine a characteristic of each target species in the sample. For example, the quantity, such as the concentration or affinity, of two or more species in the polydisperse sample can be determined. A series of dilution steps, which comprises a minimum of three dilutions, can be performed of the polydisperse sample at two or more probe concentrations. Preferably, one dilution is above, one is around and one is below the expected K_{D} of the species. The accuracy of the results will be improved by having more than three dilutions in the series: typically 5, 6, 7, 8 or more dilutions are used to order to cover a reasonable range with reasonable accuracy. By adding a probe into the polydisperse sample, the probe can be configured to bind to two or more species to form a probe-target species complex.

The next step of the workflow is to separate two or more species at each dilution step into two or more bins by any separation technique such as diffusion. In some instances, separating two or more species at each dilution step into two or more bins may utilise a separation device, for example a capillary electrophoresis device. Capillary electrophoresis can be deployed to separate a sample into multiple, separated species, for example isoforms of a protein, such as monomers, dimers, oligomers or different e.g. post-translational modifications.

Electrophoresis is a technique for separation of nucleic acids, peptides, and cells. Gel electrophoresis, in which analyte charge-to-size ratio is assessed via retardation in a solid matrix upon the application of an electric field, is the most common technique, though this is not well suited for the study of weak protein association events as the act of matrix sieving itself can disrupt interactions. Capillary Electrophoresis (CE) involves the temporal separation of analytes based on their differential electrophoretic mobility and electroosmotic flow throughout a channel. In Free-Flow Electrophoresis (FFE), the sample moves throughout a planar channel through pressure or displacement-driven flow, and separation upon application of an electric field is perpendicular to the direction of flow. Because FFE is a steady-state technique, injection and separation are performed continuously. Microfluidic Free-Flow Electrophoresis (MFFE), a microfluidic miniaturization of FFE, has the advantage of improving separation resolution by reducing the effect of Joule heating and facile on-line integration with other separation techniques.

The separation of the species using capillary electrophoresis is made using the mobility differences of the two or more species such as different protein complexes with labelled affinity probes and/or immunoprobes. Different peaks should appear in function of the mobility of the different type of biomolecule within the solution.

The separation step may be performed under native conditions to allow an understanding of the species and its environment, including its relationship with other species in a multicomponent mixture. The subsequent analysis may include denaturing and labelling steps to permit accurate identification and characterisation of separated component.

A characteristic of each of the two or more species in each bin can be detected using high resolution or high sensitivity detection techniques such as a fluorescence detection technique by means of a photodiode, a single photon avalanche diode (detector) (SPAD), a photomultiplier or a high resolution camera.

Thus, the concentration of each separated probe-target species complex in each bin at each dilution step can be determined using a curve fitting model. This means that the quantitative information about the biophysical properties of the species such as proteins and/or protein-protein interactions can be obtained. For example, the concentration of the free probe and the concentration of all separated sub-species of proteins and protein complexes can be obtained. Moreover, the quantity of each target species in the sample and/or the affinity of each target species to the probe of the sample can be determined using a binding model, as disclosed in the Examples.

Referring to FIGS. 1A to 1B, there is shown plots of experimentally obtained electrophoretic separations at different probe concentrations. As illustrated in FIGS. 1A to 1B, a dilution series is performed on the sample and two or more species at each dilution step is electrophoretically separated. FIG. 1A, shows the experimental data obtained at 75 nM probe concentration. FIG. 1B shows the experimental data obtained at 25 nM probe concentration. From FIGS. 1A and 1B, the probe-target affinities and concentrations of each species in the sample i.e. each peak or each mobility value can be obtained.

Referring to FIG. 2, there is provided an example of a plot showing a dilution series of one separated species in order to determine the concentration and affinity of that species to the probe. This corresponds to an exemplary workflow to be tested on a sample that may contain isoforms of alpha synuclein and serum. By way of example only, FIG. 2 illustrates that two dilution series are prepared by incubating several dilutions of the sample with different concentration of affinity probes. For example, the concentration of the probe can be 17 nM and 75 nM as illustrated in FIG. 2. The species within the sample can be characterised with the dilution series at different probe concentrations to extract both the dissociation constant and the concentration of the species. The difference of the measured concentrations as shown in FIG. 2 can give the size of the subspecies. The data can be fitted globally to minimise bias.

Referring to FIG. 5, there is shown a flowchart illustrating the workflow 10 of the present invention. The method as disclosed herein can be used to determine the quantity of two or more species in a polydisperse sample 12. The method of the present invention includes the step of performing a series of dilution steps of the polydisperse sample 12. One or more probes 13 can then be added into the polydisperse sample 12, where the probe 13 is configured to bind to two or more species to form two or more probe-target species complexes 14. There are advantages to performing the dilution steps before adding the probe 13 in that the same probe stock can be used and applied to each dilution. However, the steps are not necessarily carried out in the order listed above.

The next step of the workflow is to separate two or more species at each dilution step 16 into two or more bins 20, 22 by any separation technique 18 such as diffusion. Additionally or alternatively, the separation technique 18 can also be, but is not limited to, electrokinetic separation technique. In some instances, separating two or more species at each dilution step into two or more bins may utilise a separation device, for example a capillary electrophoresis device.

A detector 24 is provided to detect the two or more species bound to a probe separated into two or more bins 20, 22 at each dilution step. The detection techniques deployed may be selected, as appropriate from various different approaches to the detection of electromagnetic radiation of various types. The detection technique may include, but is not limited to, optical detection techniques. The assumption is that the probe can be detected, either free or in complex with the target species, and that different complexes can be separated. As shown in FIG. 5, the concentration of each separated probe-target species complex is then determined in each bin 20, 22 at each dilution step. In addition, the quantity of each target species in the sample and/or the affinity of each target species to the probe is also determined.

Referring to FIG. 5, the workflow 10 includes a feedback mechanism 26 where further experiments are suggested or recommended based on results from previous experiments. The choice of which probe concentration 28 and/or which sample dilution 30 to use during experiments can either be considered before the experiment or updated during the experiment by using a feedback mechanism 26 based on results from previous experiments. Choosing the most suitable dilution 30 or probe concentration 28 can be from a user designing the experiments, or it can be chosen automatically by using automated software after analysing the results of previous experiments. The analysis of the results may be based on Bayesian analysis.

### EXAMPLES

### Single Binding Partner

In one example, the method of the invention as disclosed herein can be used to investigate a single binding partner to the species of interest.

The interaction between a probe A and a target B is described by the dissociation constant (K_{d}). The dissociation constant is defined as the ratio of concentrations between the free target ([B]) and probe ([A]) with the complex formed by the interaction between these two ([AB]):

$$K d = \frac{[ A ] [ B ]}{[ A B ]}$$

The total concentrations of probe ([A₀]) and target ([B₀]) are given by:

$$[ {A}_{0} ] = [ A ] + [ A B ]$$

$$[ {B}_{0} ] = [ B ] + [ A B ]$$

In one example, the tight binding regime can be characterised by the dissociation constant being much lower than the probe concentration (K_{D}<<[A₀]). All the target particles are bound provided enough probe particles are available. This is very useful to measure the concentration of target as the measured quantity is the target concentration ([AB]≈[B₀]). This is only true in the tight binding regime (K_{D}<<[A₀]) when the probes are not depleted ([B₀]<[A₀]). If the probes are depleted, the sample needs to be diluted adequately to avoid saturation. In this regime, the dissociation constant cannot be measured. The probe would therefore need to be diluted to reach the soft binding regime.

In a further example, the soft binding regime can be characterised by the dissociation constant being on the same order of magnitude as the probe concentration (K_{D}≈[A₀]). In this regime, the measured complex concentration is a function of the target concentration and dissociation constant ([AB]=[AB]([B₀], K_{D})). By performing several experiments at different dilutions of either the probe and/or the target, this function can be fitted, yielding the concentration of targets and their dissociation constant. The number of required experiments is lowered if one of these values is already known. If the concentration of targets is low ([B₀]<<[A₀]), the function can be approximated to the first order ([AB]([B₀], K_{D})≈[B₀]/(K_{D}+[A₀])). If there is a large excess of targets ([B₀]>>[A₀]), the system is saturated ([AB]≈[A₀]) and the sample needs to be diluted.

In another example, the non-binding regime can be characterised by the dissociation constant being much larger than the probe concentration (K_{D}>>[A₀]). In this case, most targets are unbound and no useful information on the interaction can be extracted.

### Multiple Binding Targets

Commonly available biophysical techniques are usually used to determine the binding affinity for just one target species. If there are two or more target species, the standard way of determining affinity via K_{D}⁽ⁱ⁾=[free probe]×[free target⁽ⁱ⁾]/[target complex⁽ⁱ⁾], using [total target⁽ⁱ⁾]=[free target⁽ⁱ⁾]+[target complex⁽ⁱ⁾] for each species i individually as another fit variable, does not work since the species compete with each other for the probe. Thus, an improved binding model may be used to determine the binding affinity of two or more species.

In the case of multiple targets Bᵢ, the binding equations are:

$${K}_{D , i} = \frac{[ A ] [ {B}_{i} ]}{[ A {B}_{i} ]}$$

$$[ {A}_{0} ] = [ A ] + {\sum }_{i} [ A {B}_{i} ]$$

$$[ {B}_{0 i} ] = [ {B}_{i} ] + [ A {B}_{i} ]$$

If the concentration of targets is small (Σᵢ[B₀ᵢ]<<[A₀]), the probe is not significantly depleted ([A]≈[A₀]). Therefore, the above discussion for the single binding partner applies. For example, in the strong binding case (K_{D,i}<<[A₀]), all the targets of the species i are bound ([ABᵢ]≈[B₀ᵢ]). In the soft binding case (K_{D,i}≈[A₀]) the complex concentration is a linear function of the target concentration ([ABᵢ]≈[B₀ᵢ]/(K_{D,i}+[A₀])).

If the concentration of targets is comparable to the probe concentration (Σᵢ[B₀ᵢ]˜[A₀]), competitive binding comes into play. When the concentration of targets is very high (Σᵢ[B₀ᵢ]>>[A₀]), the probe is depleted ([A]<<K_{D,i}). In that regime, the concentrations are controlled by the concentration over dissociation constant ratio:

$$[ A {B}_{i} ] \approx \frac{[ {B}_{0 i} ]}{{K}_{D , i}} / {\sum }_{j} \frac{[ {B}_{0 j} ]}{{K}_{D , j}} .$$

An example is illustrated in FIG. 3.

As illustrated in FIG. 3, there are some surprising observations from the results including the curves crossing over at a point on the graph and also, that they are not monotonic. This can be explained by the transition from the low target concentration regime where [B₀ᵢ]/(K_{D,i}+[A₀]) is the controlling factor to the high target concentration regime where [B₀ᵢ]/K_{D,i} is the controlling factor. Unlike the single target case, the dissociation constant plays a role even with tight binding, provided the targets concentrations are large enough. The model here assumes that the targets only interact through competition for the probes. This model does not consider interactions between targets.

### Measuring Concentration and/or Dissociation Constant

As disclosed in the present invention, the concentration and/or the dissociation constant of target species can be measured. The assumption is that the probe can be detected, either free or in complex with the target species, and that different complexes can be separated. One approach of doing this is by attaching fluorescent labels to the probe. A chromatography technique, such as capillary electrophoresis, can be used to separate the different complexes.

In some instances, a probe is labelled with a fluorophore. Several solutions can be prepared by mixing different concentrations of labelled probe, sample, and dilution buffer. Each of these solutions is given sufficient time to incubate and reach equilibrium. Then, these solutions are loaded in a capillary electrophoresis (CE) device where they undergo separation and detection.

The detected fluorescence signal typically comprises several parts:
Fluorescent signal from the labelled probes;
Non-binding signal from the labelled probes;
Fluorescent signal from the sample: The sample can be fluorescent. In this case, running a solution with no labelled probes reveals the fluorescence signal that can then be subtracted.
Background signal: The camera might have some electrical noise. The chip or capillary might be slightly fluorescent, as could the running buffer. There might be some external light that can leak in. These backgrounds can be dealt with by background subtraction. If they are time independent, measuring the intensity in the CE channel before the run is sufficient to remove any contribution. If they are time dependent, a second detection point can be set up next to the channel to track the background variations over time.

After having extracted the probe fluorescence over time from the signal, it can be converted to concentration as a function of electrophoretic mobility. The concentration by unit length is obtained from the fluorescent signal by knowing the fluorophore grayscale rate, the injection volume, the detection volume, and the cross section of the detection channel. From there the concentration per mobility unit is obtained by multiplying by the detection time and the electric field.

The signal of the probe without any sample, or very heavily diluted sample, can be used to subtract the amount of unreacted probes from each measurement. This is particularly useful if the free probes are not fully separated from the bound probes. This can also be used to quantify the amount of bound probe.

An example of concentration per unit mobility plot is shown in FIG. 4. FIG. 4 illustrates the separations of subspecies at different sample concentrations and constant probe concentration.

In some cases experimental conditions may vary and therefore, it is key to keep conditions similar. The curves, as illustrated in FIG. 4, must be taken in similar conditions to be compared. Some variations may include:
Electro-osmotic flow: Changing the ionic composition of the solution might change the zeta potential on the wall of the capillary, therefore leading to concentration-dependent electro-osmotic flow.
Pressure/Voltage: The external conditions could vary because of changing capillary property over time or because of a bubble stuck somewhere in the system.
Off-target interactions: If the sample contains multiple proteins, some of them might create off-target interactions. Similarly, there has been observations of interactions with the free dye left over from the probes labelling.
Wall absorption: Coating can be used to minimise wall absorption.

All variations that change the interactions are not detectable, unless they are time dependent. Variations that affect the mobility can be corrected by comparing similar runs. For example, for the case of a dilution curve, each dilution can be compared and aligned to the previous dilution.

The next step is to identify the peaks in the electropherogram corresponding to each species. The probe peak can usually be identified in a dilution series as it decreases with increasing sample concentration. Ideally, each peak would be completely separated. In this case, the concentration can simply be integrated over each peak to extract the species concentration. If the peaks are only partially separated, other techniques such as Gaussian fitting of the peaks may be used for separating the species. Finally, if the peaks are completely overlapping, a separation may still be possible by adding the species ratio as a fitting parameter. This would however lead to a large error as separating these two species would be difficult. Any separation of partially or completely overlapping peaks is only possible if the concentration of the two peaks is similar. If a peak is much more concentrated than the other one, the signal of the second will be hidden in the noise of the first. In this case a complete separation is required.

### Variations on the Technique

Several other parameters could be varied, such as the pH or the temperature. This could be useful to measure variations of the size or dissociation coefficient with respects to these parameters.

Running a partially incubated sample might give information on the kₒₙ and k_{off} parameters. For example, a time series can be carried out on a given sample and record the variation of concentrations.

If the isoforms are in a dynamic equilibrium, as monomers and oligomers of a protein might be, running while varying the pH or temperature might bring interesting information. Similarly, a time series could bring information on the aggregation dynamics.

Various further aspects and embodiments of the present invention will be apparent to those skilled in the art in view of the present disclosure.

“And/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A method of determining the quantity of two or more species in a polydisperse sample, the method comprising the steps of:
performing a series of dilution steps of the polydisperse sample;
adding a probe into the polydisperse sample, wherein the probe is configured to bind to two or more species to form two or more probe-target species complexes;
separating two or more probe-target species complexes at each dilution step into two or more bins;
determining the concentration of each separated probe-target species complex in each bin at each dilution step; and
determining the quantity of each target species in the sample and/or the affinity of each target species to the probe.

2. The method according to claim 1, wherein the method steps are carried out within a diluted regimen.

3. The method according to claim 1 or 2, wherein the step of determining the quantity of each target species in the sample includes fitting the measured probe-target species complexes to a model including binding equilibria with one or more affinities between probes and targets, where the fitting parameters are the concentrations of each target and their affinities to the probe.

4. The method according to any one of the preceding claims, wherein the method further comprises the step of determining the quantity of free probe present in each bin.

5. The method according to any one of the preceding claims, wherein the same concentration of probe is added to each dilution step.

6. The method according to any one of the preceding claims, wherein the concentration of the probe added to one or more of the dilutions steps differs from the concentration of the probe added to one or more of the other dilutions steps.

7. The method according to any one of the preceding claims, further comprising the step of selecting the concentration of the probe and/or selecting one or more dilutions step using a feedback mechanism based on the determined concentration of each separated probe-target species complex, the determined quantity of each target species and/or the affinity of each target species to the probe obtained in a previous experiment.

8. The method according to any one of the preceding claims, wherein the probe is an affinity probe.

9. The method according to claim 8, wherein the affinity probe is an antibody, antibody fragment, a peptide, a protein scaffold, an affimer, an affibody, a single-chain antibody, a single-chain variable fragment (scvf), a small molecule, a nanobody, an aptamer or a darpin.

10. The method according to any one of the preceding claims, further comprising the step of labelling the probe with a label.

11. The method according to claim 10, wherein the label is at a single binding site on the probe.

12. The method according to claim 11, wherein the label is a fluorescent molecule.

13. The method according to any one of the preceding claims, wherein the probe has a lower molecular weight than the target species.

14. The method according to any one of the preceding claims, wherein the separation of the two or more species is created electrophoretically through the application of an electric field.

15. The method according to any one of the preceding claims, wherein the separation of the two or more species is created by capillary electrophoresis.

16. The method according to any one of claims 13 to 15, further comprising the step of identifying one or more, partially or completely, separated peaks corresponding to each species.

17. The method according to claim 16, further comprising the step of applying a Gaussian fitting to the partially separated peaks.

18. The method according to any one of the preceding claims, further comprising the step of removing background signal of the probe.

19. The method according to any one of the preceding claims, wherein the species is an antibody or antibody fragment thereof, a polypeptide, a polynucleotide, a protein, a small molecule or a polysaccharide.

20. The method according to any one of the preceding claims, further comprising the step of adjusting one or more parameters of the polydisperse sample to facilitate binding between the probe and the two or more species.

21. The method according to claim 20, wherein the parameters is one or more of the following: temperature, salt concentrations, viscosity of the sample or surfactant concentration.
